(19) United States
(12) Patent Application Publication
(10) Pub. No.: US 2014/0276111 A1
(43) Pub. Date: Sep. 18, 2014

(54) LOW COST MEDICAL IMAGING SYSTEMS AND METHODS

(71) Applicant: CALCULA TECHNOLOGIES INC., San Francisco, CA (US)

(72) Inventors: David Gal, San Francisco, CA (US); Buzz Bonneau, San Francisco, CA (US)

(73) Assignee: CALCULA TECHNOLOGIES INC., San Francisca, CA (US)

(21) Appl. No.: 14/208,026

(22) Filed: Mar. 13, 2014

Related U.S. Application Data
(60) Provisional application No. 61/800,200, filed on Mar. 15, 2013.
(60) Provisional application No. 61/857,990, filed on Jul. 24, 2013.

(51) Int. Cl. A61B 1/00; A61B 1/04; A61B 5/00

(52) U.S. Cl. CPC A61B 1/00167; A61B 5/0084; A61B 1/04; A61B 1/0002; A61B 1/00009; A61B 1/00055
USPC 600/478

(57) ABSTRACT

A medical imaging system, the medical imaging system may include a non-coherent fiber bundle that comprises multiple fibers; wherein each of the multiple fibers has a distal end and a proximal end; at least one lens optically coupled to the non-coherent fiber bundle; an imaging sensor that is arranged to receive light received from the non-coherent fiber bundle and to generate detection signals; and a non-volatile memory module that stores mapping information that associates between locations of distal ends and proximal ends of the multiple fibers.

### RELATED APPLICATIONS

This application claims priority from U.S. provisional patent Ser. No. 61/800,200 filing date Mar. 15, 2013 and from U.S. provisional patent Ser. No. 61/857,990 filing date Jul. 24, 2013 both being incorporated herein by reference.

### BACKGROUND

Visualization of tissues, structures and tools in medical practice is often critical to a successful clinical outcome. During traditional open surgeries and procedures this was relatively trivial—the practitioner simply looked into the body. With the advent of minimally invasive and endoscopic procedures, however, advances in visualization have become necessary to properly view the surgical field. To that end advances in visualization technology have paralleled the miniaturization of surgical tools and techniques.

The primary way to directly visualize an endoscopic procedure is to insert a camera into the field and observe on a monitor. The two primary embodiments used for in-vivo cameras are “chip-on-stick” and fiber optics. Chip-on-stick refers to the use of a CMOS or CCD sensor at the distal end of a medical instrument. The sensor converts the image (light) signal into an electrical signal, which is transmitted to the proximal end of the medical instrument. Fiber optic cameras utilize several optical fibers (usually several thousand) to transmit light via the principle of total internal reflection to a sensor or eyepiece on the proximal end of the medical device. Each fiber with in the bundle is effectively a “pixel” in a spatially sampled image.

Fiber cameras currently have a larger market share than chip-on-stick technology. This is due to the relative nascency of chip-on-stick technology. Generally speaking, chip-on-stick provides a higher quality image and a theoretical lower price point but are typically larger than fiber based solutions. Fiber optic solutions are generally required when a camera cross-sectional area below 1 mm²is desired.

Today's fiber optic cameras are all based on a coherent bundle—several thousand fibers bundled together such that all fibers are parallel to one another. The implication of this is that a fiber in a given location on one end of the bundle will match to a known and corresponding location on the opposite end.

Without the arrangement of a coherent bundle the resultant image on the distal end would be scrambled (e.g. the pixels wouldn't be in their correct relative locations).

A typical image obtained using a bundle of fibers includes voids (gaps)—“interstitial space.” The greater the number of fibers, the less severe and obvious the voids in the image are. This is due to the fact that a bundle with N fibers magnified to a spot size of diameter D will have relatively larger fibers (and interstitial voids) than a bundle with 2N fibers magnified to a spot size D. Many endoscopes contain fiber cameras that provide much higher fidelity images than shown above.

One major issue with today's fiber cameras is that they are expensive. One of the leading drivers associated with the cost of fiber optic cameras is the coherent fiber bundle itself. There is significant cost and knowhow associated with the manufacture of such fiber bundles. It is often prohibitive for small manufacturers and companies to make their own bundles. They're restricted to purchasing from the limited number of global companies that make coherent bundles. These companies specialize in fiber manufacturing and charge a significant premium for their fibers. $50-$500/meter of fiber is typical.

A second major cost associated with traditional fiber optic imaging systems is the proximal lens system, which magnifies the proximal face of the fiber bundle onto an imaging sensor or eyepiece. It is not uncommon for such lens systems to require 3 or more lenses and cost $100 or more. In addition to the cost associated with the lenses, the proximal magnification system requires a relatively precise mechanical housing and takes up space and adds weight to the device.

It is often advantageous to design a medical device to be disposable. This simplifies the design (the device no longer needs to be resterilizable) and eliminates the reprocessing time. The medical facility, for example, does not need to bother with sterilization and can instead simply dispose of the product at the end of the procedure.

In the United States, insurance companies reimburse doctors and facilities for medical procedures. Generally speaking there is a fixed rate of reimbursement for a given procedure. Any costs associated with said procedure—including the cost of devices used—must be less than the reimbursed amount if the facility and doctor is to make a profit. To that end fiber cameras that have a raw fiber cost of $50-$500/meter and or proximal lensing systems that cost a few hundred dollars are prohibitively expensive for most disposable medical applications.

Many medical procedures and devices would benefit from direct visualization, but do not necessarily require the fidelity provided by modern chip on stick or high resolution fiber bundles. Cannulation of a body lumen, confirmation of device location, and identification of an aberration within a body lumen are all examples of situations that may benefit from visualization, but might not actually require the fidelity rendered by a modern and expensive endoscope and camera system. Clinical examples of such scenarios include, but are not limited to locating a kidney stone within a ureter, identifying the vocal cords during intubation, and identifying a blockage in a fallopian tube. The above examples hardly require a high-resolution image.

### SUMMARY

According to an embodiment of the invention there may be provided a medical imaging system may include a non-coherent fiber bundle that may include multiple fibers; wherein each of the multiple fibers has a distal end and a proximal end; at least one lens optically coupled to the non-coherent fiber bundle; an imaging sensor that may be arranged to receive light received from the non-coherent fiber bundle and to generate detection signals; and a non-volatile memory module that may store mapping information that associates between locations of distal ends and proximal ends of the multiple fibers.

The at least one lens may include a proximal lens that is optically coupled to the non-coherent fiber bundle.

The at least one lens may include a distal lens fixed to the non-coherent fiber bundle.

The imaging sensor is adhered to the non-coherent fiber bundle.

The non-coherent fiber bundle and the at least one lens belong to a disposable portion of the medical imaging system.

The non-volatile memory module may store information about transfer properties of the multiple fibers.

The non-volatile memory module may store information about malfunctioning fibers of the multiple fibers.

The medical imaging system may include an image processor that may be arranged to receive the detection signals and the mapping information and to reconstruct at least a portion of an image of an object that faces the distal end of the multiple fibers.

The non-volatile memory module may store information about transfer properties of the multiple fibers; and wherein the image processor may be arranged to modify the at least portion of the image in response to the information about transfer properties of the multiple fibers.

The non-volatile memory module may store information about malfunctioning fibers of the multiple fibers; and wherein the image processor may be arranged to reconstruct the at least portion of the image in response to the information about malfunctioning fibers of the multiple fibers.

The image processor may be arranged to compensate for gaps between the multiple fibers.

The image processor may be arranged to compensate for a gap formed between adjacent fibers of the multiple fibers by performing interpolations between a subset of pixels out of all pixels associated with the adjacent fibers.

The subset of pixels may include a single pixel per fiber.

The image processor may be arranged to digitally magnify the image of the object.

According to an embodiment of the invention there may be provided a medical imaging system that may include a non-coherent fiber bundle that may include multiple fibers; wherein each of the multiple fibers has a distal end and a proximal end; at least one lens optically coupled to the non-coherent fiber bundle; an imaging sensor that may be arranged to receive light received from the non-coherent fiber bundle and to generate detection signals; and an image processor that may be arranged to receive the detection signals and reconstruct an image of an object that faces the distal end of the multiple fibers in response to the detection signals and to mapping information that associates between locations of distal ends and proximal ends of the multiple fibers.

The at least one lens may include a distal lens that is optically coupled to the non-coherent fiber bundle.

The at least one lens is fixed to the non-coherent fiber bundle.

The image processor may be arranged to calculate the mapping information.

The image processor may be arranged to calculate the mapping information in response to information about malfunctioning fibers of the multiple fibers.

The image processor may be arranged to calculate the mapping information in response to an expected content of the image.

The image processor may be arranged to calculate the mapping information in response to an expected content of a calibration image obtained when imaging a calibration target.

The non-coherent fiber bundle and the at least one lens belong to a disposable portion of the medical imaging system.

The medical imaging system may include a non-volatile memory that may store the mapping information.

The non-volatile memory module may store information about transfer properties of the multiple fibers.

The non-volatile memory module may store information about malfunctioning fibers of the multiple fibers.

The image processor may be arranged to receive the detection signals and the mapping information and to reconstruct an image of an object n object that faces the distal end of the multiple fibers.

The image processor may be arranged to reconstruct the at least portion of the image in response to the information about transfer properties of the multiple fibers.

The image processor may be arranged to reconstruct the at least portion of the image in response to the information about malfunctioning fibers of the multiple fibers.

The image processor may be arranged to compensate for gaps between the multiple fibers.

The image processor may be arranged to compensate for a gap formed between adjacent fibers of the multiple fibers by performing interpolations between a subset of pixels out of all pixels associated with the adjacent fibers.

The medical imaging system 4 wherein the subset of pixels may include a single pixel per fiber.

The image processor may be arranged to digitally magnify the image of the object.

The medical imaging system further may include a light source and wherein at least one fiber of the multiple fibers is utilized for conveying light from the light source.

According to an embodiment of the invention there is provided a method that may include directing light from an object, through at least one lens and a non-coherent fiber bundle and onto an imaging sensor; wherein the non-coherent fiber bundle may include multiple fibers; wherein each of the multiple fibers has a distal end and a proximal end; generating, by the imaging sensor, detection signals; and reconstructing at least a portion of an image of an object that faces the distal end of the multiple fibers; wherein the reconstructing is responsive to the detection signals and to mapping information that associates between locations of distal ends and proximal ends of the multiple fibers.

The reconstructing of the at least portion of the image is also responsive to information about transfer properties of the multiple fibers.

The reconstructing of the at least portion of the image is also responsive to information about malfunctioning fibers of the multiple fibers.

The reconstructing of the at least portion of the image may include compensating for gaps between the multiple fibers.

The reconstructing of the at least portion of the image may include compensating for a gap formed between adjacent fibers of the multiple fibers by performing interpolations between a subset of pixels out of all pixels associated with the adjacent fibers.

The subset of pixels may include a single pixel per fiber.

The reconstructing of the at least portion of the image may include digitally magnifying the image of the object.

The reconstructing of the at least portion of the image occurs in real time.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 illustrates an incoherent bundle of fibers;

FIG. 2 illustrates images obtained at the proximal and distal ends of the non-coherent fiber bundle;

FIGS. 3A-3C illustrate voids that are formed between fibers or between coverage areas of fibers, and a pixel reconstruction process according to an embodiment of the invention;

FIG. 4 illustrates malfunctioning fibers of an incoherent bundle of fibers;

FIGS. 5A-5E illustrate systems according to various embodiments of the invention;

FIG. 6 illustrates various image processing stages executed by the image processor of FIGS. 5A-5E according to various embodiments of the invention;

FIG. 7A-7B illustrate the mapping between distal and proximal ends of an incoherent fiber bundle and the remapping process between distal and proximal ends of a bundle of incoherent fibers using a subset of pixels associated with a fiber for shuffling the received image data in order to realize a cogent image; and

FIG. 8 illustrates a method according to an embodiment of the invention.

### DETAILED DESCRIPTION OF THE DRAWINGS

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

Because the illustrated embodiments of the present invention may for the most part, be implemented using electronic components and circuits known to those skilled in the art, details will not be explained in any greater extent than that considered necessary as illustrated above, for the understanding and appreciation of the underlying concepts of the present invention and in order not to obfuscate or distract from the teachings of the present invention.

Any reference in the specification to a method should be applied mutatis mutandis to a system capable of executing the method and should be applied mutatis mutandis to a non-transitory computer readable medium that stores instructions that once executed by a computer result in the execution of the method.

Any reference in the specification to a system should be applied mutatis mutandis to a method that may be executed by the system and should be applied mutatis mutandis to a non-transitory computer readable medium that stores instructions that may be executed by the system.

Any reference in the specification to a non-transitory computer readable medium should be applied mutatis mutandis to a system capable of executing the instructions stored in the non-transitory computer readable medium and should be applied mutatis mutandis to method that may be executed by a computer that reads the instructions stored in the non-transitory computer readable medium.

The terms “system”, “apparatus”, “medical imaging system” are used in an interchangeable manner.

The term “bundle” refers to an incoherent bundle unless expressly stated that the bundle is coherent.

There is provided a low cost direct visualization method, system, and device for visualizing body lumens. In particular we present methods of using lower cost incoherent fiber to realize an image as well as methods for enhancing images captured with fiber optic systems.

As explained above a coherent fiber bundle translates image data intact since all fibers are parallel to each other. As such each fiber samples a portion of the image and transmits it to the corresponding location on the proximal end of the bundle. If the proximal end of the fiber bundle is imaged (e.g. a picture is taken of the proximal portion of the bundle) the image at the distal end is seen reflected across the y-axis.

An incoherent bundle, however, has a random relative arrangement of fibers within the bundle. This is to say that given a fiber at one end of the bundle there is no way to identify its corresponding location on the other end by visual inspection alone. Typically incoherent bundles are used for illumination or energy delivery—applications wherein the precise relative positioning of the fibers is inconsequential.

FIG. 1 illustrates a prior art in-coherent bundle of fibers 10 that includes multiple fibers 10(1)-10(K). The in-coherent bundle of fibers 10 includes a proximal end 11 and a distal end 12. The proximal ends and the distal ends of the multiple fibers that form the bundle are located at the proximal end 11 and distal end 12 respectively.

The location of a distal end and the proximal end of a same fiber (of the multiple fibers) may differ from each other. Letters a,b,c,d, represents distal and proximal ends of four fibers. They are located at different corresponding locations.

As a result the use of incoherent bundle would result in a scrambled image as seen in FIG. 2. Image 13 illustrates an image of a person (as viewed from the distal end 12 of the bundle) while image 14 illustrates the image that is provided at the proximal end 11 of the bundle. Due to the difference in locations between distal and proximal ends of the fibers 10(1)-10(k) the image 14 formed at the distal end 12 does not resemble a person.

Incoherent fiber bundle typically cost an order of magnitude less than coherent fiber. Significant systems cost savings could be accomplished if it were possible to utilize incoherent fiber for imaging purposes.

Because the ends of the fiber bundle are fixed (e.g. the configuration of the fibers at either end of the bundle remains unchanged) there exists a unique mapping from one end to another. In other words for a given incoherent bundle the captured image will always be scrambled in the same way.

Given a mapping from one end of the bundle to the other it would be relatively straightforward to unscramble the image and properly reconstruct it. The mapping would effectively serve as a lookup table and the image data could be shifted accordingly.

The mapping itself is also relatively straightforward to realize. Using known images some and basic algorithms it is straightforward to compare the expected (e.g. original image) with the realized (e.g. received) image. The most naïve algorithm, which would never be used due to computational time, is simply an iterative one, which tries all possible mappings until the correct one is found (e.g. by comparing the mapped image to the original until there is a match). More realistic algorithms include using a known gradient pattern such that each fiber collects a unique and known light spectral information and intensity. By collecting and comparing the scrambled wavelength and intensities to the original gradient pattern the proper mapping could be realized efficiently.

It should be understood that this mapping is actually performed on the image sensor pixel level. The implication is to utilize a complete fiber imaging system with an imaging sensor. The mapping would be performed per pixel of the imaging sensor. In other words the image sensor's pixels would be shuffled around in order to realize the desired output image.

There are other mappings that may facilitate image reconstruction and enhancement. The individual fibers in a bundle (coherent or incoherent) are extremely delicate. They can often break during manufacturing, during shipment, or during use. These dead fibers typically show up as aberrations in the image. In the extreme case where the fiber is broken such that it transmits no light a black spot would visible where light would have otherwise excited the image sensor. FIG. 4 illustrates distal and proximal ends 19(1)-19(4) and 19′(1)-19′(4) respectively of four dead fibers. Typically when a threshold level of fibers are broken the fiber is discarded. If the fibers break during manufacturing the manufacturer must discard the product at a loss. If they break during use the physician must discard the product at a loss. The ability to ameliorate the effects of broken fibers would be highly beneficial to both the product manufacturer and the end user. A dead fiber (e.g. broken fiber) map, for example, could help facilitate both the spatial mapping process as well as the reconstruction.

Mapping the dead pixels before the spatial mapping process described above would facilitate the spatial mapping process since the image sensor pixels associated with dead fibers could simply be ignored. Additionally, during the image reconstruction phase the image sensor pixels associated with the area covered by the dead fibers could be interpolated by surrounding pixels associated with other functional fibers thus filling in a “best guess” for the voids or aberrations in the resulting image. This could be useful in applications where a few broken fibers do not result in a clinically useless image, but are rather distracting and annoying to the user.

Another interesting mapping is the relative intensity of each fiber. It should be expected that during manufacturing not all fibers within the bundle are created equally. Some fibers will likely be more efficient at passing light than others. In order to correct for this an intensity normalization map can be realized such that the resultant image can be normalized appropriately to mitigate the effects of a “hot” or “cold” fiber. Capturing a single wavelength and intensity (e.g. an image that's uniformly bright—a Lambertian surface for example) across all fibers and measuring the light output on the proximal end (e.g. imaging the proximal end) would facilitate the generation of a lookup table or map, which contains the relative efficiency of light transmission of the fibers in the bundle. One possible way to generate this map is to image the Lambertian surface and calculate the average imaging sensor pixel value associated with each fiber in the bundle. In an ideal world wherein all fibers are identical and all pixels in the imaging sensor are identical one would expect that the aforementioned pixel averages be identical for all fibers. In reality there will be some amount of deviation for reasons explained above. A canonical method of realizing a normalizing map is to divide the averages by the maximum average. The collected pixel values could then be normalized in real-time by the realized by the normalization map. Multiple input wavelengths could be used in order to realize a more complete mapping e.g. the relative efficiency of transmission of different wavelengths could provide a better representation of transmission efficiency than a single wavelength. Collecting this data in a look up table would allow for ‘on the fly’ or post-processing correction.

FIGS. 3A-3C illustrates voids (such as voids 15(1)-15(3) of FIG. 3A) that are formed between fibers (or between coverage areas of fibers). This is the result of spatial sampling using circular fibers. When the circular bundles are squeezed together there are inevitably interstitial spaces formed due to a packing factor that is less than one. For simplicity FIGS. 3A-3C illustrate a rectangular packing arrangement, but it should be appreciated that any packing arrangement is possible. A hexagonal packing arrangement, for example, is typical of commercial fiber bundles and would nonetheless exhibit interstitial voids similar to those depicted in FIGS. 3A-3C. FIG. 3B depicts a portion of the fiber bundle being spatially sampled by an imaging sensor. The imaging sensor is shown schematically as an array of square pixels 16.

These voids are often visually unappealing and distracting. To that end another interesting real-time enhancement is to fill in the voids with interpolated image data. This is readily accomplished using, among other techniques, traditional bilinear interpolation with four nearest neighbors. Depending on the fiber packing arrangement (e.g. hexagonal) other interpolation algorithms may be advantageous. In any event information from nearby fibers can be used to interpolate the interstitial voids between fibers. As seen in FIGS. 3B and 3C the imaging sensor's pixels are represented as the square grid and the circles represent individual fibers in the bundle. The fibers are oversampled by the imaging sensor such that the Nyqvist sampling rate is satisfied. FIG. 3C illustrates the utilization of image sensor pixels 17 associated with adjacent fibers of 10(1), 10(2), 10(5) and 10(6) to interpolate a value for one of the pixels 18 in the interstitial space 15(1).

Because the above techniques (spatial mapping, intensity normalization, dead pixel mapping) inherently require mapping tables with information about the location of the fibers relative to the imaging sensor this same information can be utilized for interpolation. This is advantageous since typical void interpolation techniques use on the fly detection to locate the circular fibers. This computation is complex and time consuming.

The aforementioned spatial mappings and image enhancements are not mutually exclusive, but coupled together they can result in a nicer more pleasant image. That stated there is a non-trivial amount of computational complexity that grows linearly with fiber size and/or image sensor pixel count. A large image sensor, for example, may have several million pixels, which need to be rearranged and shuffled to map the image. Additionally a fiber bundle with more fibers clearly has larger lookup tables/maps. It should be clear, however, that these millions of pixels are effectively sampling thousands of fibers. That stated for each fiber there is a group of pixels associated with said fiber. (FIG. 3B illustrates a grid 16 of rectangular pixels, about sixteen pixels per fiber—in other words an individual fiber's footprint on the imaging sensor is roughly sixteen image sensor pixels. Sixteen is a canonical number—any amount of image sensor oversampling which satisfies the Nyquist spatial sampling requirement would suffice and the following information is equally applicable). One can assume that for any group of imaging sensor pixels associated with an individual fiber each of the pixels in the group will have roughly the same information as the other image sensor pixels in the—the data collected by the group of pixels associated with a particular fiber are largely redundant. In other words an individual fiber will carry a variety of wavelengths and intensities, which all the image sensor pixels associated with said fiber will receive more or less equally.

As a result of the aforementioned, one way to reduce computational complexity is to sample a smaller number of pixels per fiber and use these data to reconstruct the larger image.

FIGS. 7A and 7B illustrates re-mapping of a single pixel per fiber instead of shuffling all pixels of the image. The shuffled pixels are then used for reconstructing an image of arbitrary size (for example by interpolation). It is noted that more than a subset of more than a single pixel may be shuffled and used for image reconstruction and or manipulation. FIG. 7A illustrates four fibers 10(1)-10(4) of an incoherent fiber bundle with distal ends 12(1)-12(4) and proximal ends 11(1)-11(4). Image sensor 17 is shown as an array of square pixels, which sample distal ends 12(1)-12(4). Pixels 17(1)-17(4) of the image sensor are associated with fibers 10(1)-10(4) respectively. FIG. 7B illustrates re-mapping the imaged data from image sensor 17 to memory array 17′ shown figuratively as an array of square pixels which represent the data values in the memory array. Pixels 17(1)-17(4), which are associated with fibers 10(1)-10(4) of FIG. 7A are shuffled in the memory array such that their spatial relationship to each other matches the spatial relationship of distal ends 11(1)-11(4) of fibers 10(1)-10(4). Data values 17′(1)-17′(4) correspond to pixel values 17(1)-17(4), but as seen are in their proper spatial relationship. FIG. 7B further shows data value 17′(5), which is interpolated for using values 17′(1)-17′(4). The remaining data values in memory array 17′ can be interpolated for using appropriate data values. In this manner an arbitrarily sized image can be realized from a relatively small number of image sensor pixels.

Because the pixels associated for the fiber are approximately redundant the signal to noise ratio remains roughly the same and at a gross approximation information is not lost. As a result the final interpolation step can arbitrarily size the final image. The interpolation step can be performed for any desired output image (e.g. sized to a monitor display). It should be noted that sampling a subset of the image sensor pixels is equally applicable to dead pixel correction, intensity normalization, and interpolation to reduce interstitial voids.

In the ideal case the system may only need to utilize a single image sensor pixel's worth of data for each fiber in the bundle. For a bundle with 1000 fibers this would imply 1000 collected pixels. In the case of a 1 MPixel imaging sensor this is a reduction of 1000× the data and computation. Using the limited sampled data and bilinear interpolation an entire image can be realized from very little sampled data. It may be, however, beneficial to use a slightly larger group of image sensor pixels for this interpolation. An average of four pixels, for example, may help reduce any aberrations associated with the image sensor. Additionally, in the case of a color imaging sensor with a color filter array (e.g. a Bayer pattern array), it may be beneficial to use a plurality of pixels in order to demosaic the array and then use a single R, G, B triplet associated with a single pixel for the interpolation and any additional mapping.

It should be clear that several of the techniques described above do not require an incoherent fiber bundle and are applicable in the case of a coherent bundle. Interpolating to fill in voids, correcting for dead or broken fibers, and sampling only a small number of pixels per fiber are equally attributable to coherent bundles as they are incoherent.

There are two ways of creating the aforementioned mapping tables and utilizing these tables to reconstruct the image in clinical practice. The first is to have the medical practitioner participate in the mapping process. Before use, the practitioner would engage the device in a calibration step wherein the mapping tables are built, stored, and utilized. Such a step would be analogous to white balancing a camera before use in surgery. This modality has a few advantages including the ability to map and correct for any aberrations that may have occurred during product shipment, prior use, etc. The disadvantages are that it requires user engagement and time. Though the calculation of the mapping tables may be a relatively fast process medical practitioners are busy and the ability to reduce risk of user error as well as time of procedure is advantageous. The second modality is to construct and calculate mapping tables during the manufacturing of the imaging system. The mapping tables could be stored to a local memory affixed to the imaging device. A small EEPROM or flash memory on a flexible PCB, for example, could contain requisite information for aforementioned spatial mapping and image enhancement. When the practitioner assembles the system for use the data from the local non-volatile memory could be read by the rest of the imaging system and utilized for image reconstruction. The primary downsides of this technique is the inability to correct for any aberrations or defects realized during product shipment or use

A hybrid approach may also be used—some of the mapping tables may be stored prior to arrival at the clinical setting (e.g. during manufacturing) while the doctor may participate in the construction of other mapping tables as a calibration step. In a preferred embodiment the dead fiber-mapping table could be recalculated prior to each use of the camera while other mapping tables would be calculated during manufacturing. This could result in a robust, but easy to use system.

FIGS. 5A-5E illustrates various medical imaging systems 101, 102, 103, 104 and 105 according to various embodiments of the invention.

FIG. 5A also illustrates that a part of the incoherent bundle 10 and the distal lens 20 are inserted in a lumen (having a border represented by dashed line 110) and facing an object 120 within the lumen.

FIG. 5A illustrates the system 101 as including a distal lens 20, an incoherent bundle of fibers 10, a proximal lens 30, an imaging sensor 40, an image processor 50, a memory module 60 and a display 70. Some of the components (for example 20, 10, and optionally 30 and 40) can be included in a disposable or “resposable” (e.g. rated for 10 uses) portion of the system 101.

Distal lens 20 (typically a grin lens though other options including spherical and plastic are certainly possible) is coupled to incoherent fiber bundle 10. The proximal end of fiber bundle 10 is mechanically and optically coupled to an imaging sensor 40 via proximal lens 30. Proximal lens 30 may magnify, focus, or otherwise alter the resultant image onto imaging sensor 40.

Imaging sensor 40 is typically one of a CMOS or CCD imaging sensor. The output of the imaging sensor 40 is electrically coupled to an image processor 50, which has access to any one or more of the aforementioned mapping tables located in memory module 60. The image processor displays the resulting image to display 70. Image processor 50 may preform a plurality of the following operations prior to sending its final output to display 70:
a. Spatial remapping of the incoherent fiber bundle
b. Fiber efficiency normalization
c. Dead fiber mitigation
d. Interstitial void mitigation

Memory module 60 stores mapping tables that may be populated prior to use of the incoherent bundle as a calibration step.

One non-insignificant cost in a fiber optic assembly is the proximal lens stack, which magnifies the image realized by the fiber optic bundle onto an imaging sensor or eyepiece. In the simplest embodiment this might be a single grin lens, but typically a more complex lens stack is used. Typical lens stacks might involve upwards of two or three relay lenses followed by additional lensing to transfer the image to the sensor itself. These lenses can have significant cost associated with them. To that end we augment the above techniques and embodiments in order to offset the burden of optical magnification to digital algorithms. In particular we ameliorate many of the complexities of the proximal lens stack by oversampling the fiber bundle with an imaging sensor and then digitally magnifying the resulting image. This technique is, of course, bundle topology agnostic (e.g. can be used for both coherent and incoherent bundles).-This technique could potentially save a lot of cost and help make the camera disposable. Saved costs include costs associated with the lenses on the back end, but also the mechanical housing would be greatly simplified/cheaper (no need to properly space lenses, secure them in the precise location, etc, etc). Additionally the labor costs for making the camera would be reduced due to fewer complicated manufacturing steps. This would also reduce the size and weight of the system substantially which would help with integration and space-constrained applications.

In the most extreme form such a design would involve no proximal lenses at all—the imaging sensor would directly capture the imaging bundle's image. One way to do this would be to adhere the bundle's proximal end directly to the sensor. This of course leads to the potential problem that the image captured by the fiber bundle would represent a relatively small portion of the resultant image (e.g. the sensor output). This is due to the relative sizing difference between the sensor and fiber bundle. A typical bundle diameter might be on the order of 0.5 mm with an active image area of 0.3 mm while a CMOS imaging sensor might have its smallest dimension on the order of 2 mm. As a result the fiber bundle image would only comprise roughly 15% (0.3 mm/2 mm) of the size of the output image. In the case of a relatively large fiber bundle this might not be problematic since the ratio of the bundle size to the sensor size would result in a relatively large fill factor. Digital magnification of a region of interest (ROI) can, however, alleviate this issue (e.g. digital magnification of the area of the imaging sensor associated with the bundle). This digital magnification could be performed as described in the above. One or more pixels associated with the individual fibers in the bundle would be utilized to interpolate an image of arbitrary size. Given the various aforementioned maps the system utilizes correspondence between relative individual fiber location and image sensor pixel is known a priori. In this way specific image sensor pixels can be sampled as inputs to the interpolation block. Myriad digital magnification algorithms can be used to expand the relative size of the fiber spot. One obvious example is bilinear interpolation.

A variant on this theme might utilize a single lens between the fiber and the sensor. Such a lens could be useful for better focusing the resultant fiber spot on the imaging sensor. In particular because most imaging sensors have a thin sheet of glass over them having a lens to better focus the light to the actual silicon may be advantageous. Again these techniques are equally applicable to coherent and incoherent imaging.

FIGS. 5B and 5C show systems 102 and 103 respectively. In function FIGS. 5B and 5C are identical to FIGS. 5A and 5D respectively save the fact that the embodiments shown in FIGS. 5B and 5C do not utilize proximal lenses as described by the aforementioned system optimization.

FIG. 5B illustrates the system 102 as including a distal lens 20, an incoherent bundle of fibers 10, an imaging sensor 40, an image processor 50, a memory module 60 and a display 70 Some of the components (for example 20, 10, and optionally 40) can be included in a disposable portion of the system 102.

Distal lens 20 (typically a grin lens though other options including spherical and plastic are certainly possible) is coupled to incoherent fiber bundle 10. The proximal end of fiber bundle 10 is mechanically and optically coupled to an imaging sensor 40 without the use of a proximal lens. Imaging sensor 40 is typically one of a CMOS or CCD imaging sensor. The output of the imaging sensor 40 is electrically coupled to an image processor 50, which has access to any one or more of the aforementioned mapping tables located in memory module 60. The image processor displays the resulting image to display 70. Image processor 50 may preform a plurality of the following operations prior to sending its final output to display 70:
a. Spatial remapping of the incoherent fiber bundle
b. Fiber efficiency normalization
c. Dead fiber mitigation
d. Interstitial void mitigation
e. Image rescaling by interpolation to an arbitrarily sized output image

FIG. 5C illustrates the system 103 as including a distal lens 20, an incoherent bundle of fibers 10, an imaging sensor 40, a mechanical or optical or electrical coupling element 80, a memory module 90 attached to the coupling, an image processor 50 and a display 70. Some of the components (for example 20, 10, 90, 80, and optionally 40) can be included in a disposable portion of the system 103. Memory module 90 may be a non-volatile memory module supplied with the bundle.

Distal lens 20 (typically a grin lens though other options including spherical and plastic are certainly possible) is coupled to incoherent fiber bundle 10. The proximal end of fiber bundle 10 is mechanically and optically coupled to an imaging sensor 40 without the use of a proximal lens. Imaging sensor 40 is typically one of a CMOS or CCD imaging sensor. The output of the imaging sensor 40 is electrically coupled to an image processor 50, which has access to any one or more of the aforementioned mapping tables located in memory module 60. The image processor displays the resulting image to display 70. Image processor 50 may preform a plurality of the following operations prior to sending its final output to display 70:
a. Spatial remapping of the incoherent fiber bundle
b. Fiber efficiency normalization
c. Dead fiber mitigation
d. Interstitial void mitigation
e. Image rescaling by interpolation to an arbitrarily sized output image

System 104 of FIG. 5D differs from system 103 of FIG. 5C by including proximal lens 30. FIG. 5E illustrates a system 105 that includes proximal lens 20, incoherent fiber bundle 10, proximal lens 30, imaging sensor 40, image processor 50, display 70 and memory module 90 that is attached to or may be part of incoherent fiber bundle 10. The memory module 90 may be accessed by image processor 50.

Distal lens 20 (typically a grin lens though other options including spherical and plastic are certainly possible) is coupled to incoherent fiber bundle 10.

The proximal end of fiber bundle 10 of FIG. 5E is mechanically and optically coupled to an imaging sensor 40 via proximal lens 30. Proximal lens 30 may magnify, focus, or otherwise alter the resultant image onto imaging sensor 40. Imaging sensor 40 is typically one of a CMOS or CCD imaging sensor. The output of the imaging sensor 40 is electrically coupled to an image processor 50, which has access to any one or more of the aforementioned mapping tables located in memory module 90. The image processor displays the resulting image to display 70. Image processor 50 may preform a plurality of the following operations prior to sending its final output to display 70:
a. Spatial remapping of the incoherent fiber bundle
b. Fiber efficiency normalization
c. Dead fiber mitigation
d. Interstitial void mitigation
e. Image size rescaling
f. Image rescaling by interpolation to an arbitrarily sized output image

In any of the above systems memory module 90's mapping tables are populated during the manufacturing of system 104. Optionally one or more of the mapping tables in memory module 90 are augmented, modified, or updated as a calibration step prior to use. Generally memory module 60's mapping tables are calculated prior to use as a calibration step.

Any one of memory module 60 and memory module 90 may be arranged to store at least one of the following types of information:
a. Information about transfer properties of the multiple fibers—as different fibers can attenuate incoming light by different levels.
b. Mapping information that associates between locations of distal ends and proximal ends of the multiple fibers (for example—and referring to the example of FIG. 1—a mapping function may maps distal ends 11(k) to proximal ends 12(k), wherein index k ranges between 1 and K).
c. Information about malfunctioning fibers (dead fibers) of the multiple fibers (for example, referring to FIG. 4—listing 19(1)-19(4), 19′(1)-19′(4) of both).
d. Information about the relative locations of the fibers in the bundle to the pixels on the imaging sensor.

It is noted that the medical imaging system may include both memory modules 60 and 90.

It is noted that image processor 50 has read/write access to memory module 60 and 90.

It is noted that each type of information can be calculated during the manufacturing or as a calibration step before using the bundle, can be calculated without a priori knowledge during imaging of objects and/or calibration target, may be calculated in advance but updated (new dead fibers, changes in light attenuation and the like) in response to imaging results, and the like.

It is noted that the any portion of systems 101, 102, and 103 may be reusable or disposable. In preferred embodiments incoherent bundle 10, distal lens 20, optional proximal lens 30, optional memory module 90, and optionally imaging sensor may be disposable or “resposable” (e.g. rated for certain—10—number of uses).

It is noted that in a preferred embodiment image processor 50, display 70 and memory module 60 are all reusable and any combination of the remaining system components are either disposable or “resposable” ((e.g. rated for a certain—10—number of uses).

In the above embodiments the fiber bundle and distal lens is the only component in the system, which interacts directly with the patient and, therefore, enters the sterile field. As a result it is the only portion of the system that needs to be sterilized. Since, as explained above, it is advantageous to make a single use device and not have to resterilize any components, it should be clear that the only portion of the system shown in any one of FIGS. 5A-5E that needs to be disposable is the fiber and lens. Conveniently there is a relatively significant cost associated with the imaging sensor, coupling optics, imaging reconstruction, and post processing. It is advantageous, therefore, to reuse those sections of the system in order to minimize the cost of goods associated with a procedure. It should be clear, however, that the system could be made entirely disposable if desired or entirely reusable if the fiber/lens are made to be resterilizable. Additionally the disposable/reusable boundary could shift to include or exclude any of the system components seen in FIGS. 5A-5E (e.g. the imaging sensor could be made disposable in addition to the fiber and lens while the remaining components could be reusable). In a preferred embodiment the image processor, display, and coupling between the image processor and the remainder of the system are reusable while the rest of the system is “resposable” (e.g. qualified for 10 uses).

FIG. 6 illustrates various processes that can be executed by the image processor according to various embodiments of the invention. The image processor may, for example perform image reconstructions 51 followed by post-processing 52.

Additionally or alternatively, the image processor may perform a region of interest (ROI) finding or extracting 51′ followed by digital magnification and//or interpolation of the image 52″ and then continue with the image processing 53′.

Additionally or alternatively, the image processor may perform fiber transmission efficiency normalization 51″, followed by remapping 52″, followed by dead pixel correction 53″, followed by interstitial space removal/interpolation 54″ and may also perform additional image processing 55″.

The fiber transmission efficiency normalization 51″ may be responsive to information about transfer properties of the multiple fibers—as different fibers can attenuate incoming light by different levels. The normalization is aimed for compensating for differences in the transmission of different fibers.

The remapping 52″ is responsive to mapping information that associates between locations of distal ends and proximal ends of the multiple fibers. It remaps the image sensor pixels according to the mapping information in order to reconstruct the image viewed at the proximal end of the bundle 10 for example reconstructing image 13 from pixels of image 14 using the mapping between the fibers and rearranging the pixels accordingly.

The dead pixel correction 53″ is responsive to information about malfunctioning fibers (dead fibers) of the multiple fibers. This stage may include interpolating or otherwise reconstructing the image sensor pixels that should have been transferred by dead pixels based upon image sensor pixels associated with adjacent fibers in the bundle.

The interstitial space removal/interpolation 54″ may include interpolating or otherwise reconstructing the pixels from gaps between the fibers. An example is illustrated in FIG. 3C.

The above teaches novel uses of incoherent fiber bundles for imaging and detecting aberrations in body lumens. It should be clear that the parts of the proximal portion of the system could be used with either coherent or incoherent fiber. In particular the proximal lens stack and imaging sensor could be the same regardless of fiber bundle. To this end the following cost reduction techniques are applicable to both coherent and incoherent fiber optic based imaging systems. The end product can—assuming that the selling point of the product allow for it—be single use or alternatively multiuse.

With reference now to image processor 50 of FIGS. 5A-5E, mapping and image correction or enhancement could be accomplished in either a hardware or software implementation. An FPGA or DSP would be well suited for a hardware implementation. Alternatively a hybrid between hardware and software could be advantageous. Mapping, for example, could be done in hardware (e.g. an FPGA) while image scaling and enhancement could be done in software (e.g. on a DSP or CPU).

As aforementioned mapping could be performed based on all pixels on the imaging sensor or by sampling a subset (e.g. one pixel for each fiber in the bundle) and interpolating bilinearly between said pixels in order to realize an image. Clearly the latter option has more complexity, but requires smaller mapping tables and less computational complexity associated with image remapping.

All of the above embodiments could utilize fibers with core diameters on the order of between 3 and 50 microns. Larger core sizes are possible, but would reduce the overall spatial sampling frequency given a fixed bundle outer diameter. Using cores with diameters of between 3 and 50 microns allow for outer bundle diameters on the order of between 0.3 mm and 2 mm to be used without issue. These sizes would provide reasonable spatial resolution and be small enough to allow the imaging component to be embedded in a larger device or system (e.g. a catheter, steerable sheath, endoscope, etc).

According to an embodiment of the invention there may be provided a system that may include a fiber optic bundle (coherent or otherwise), distal objective lens, CMOS imaging sensor, image processing hardware/software, and monitor for display. The CMOS image sensor pixels are at least half the size of the diameter of the smallest fiber in the bundle (e.g. the CMOS imager samples the fiber bundle with a spatial frequency that is equal to or greater than the Nyqvist frequency). The proximal end of the fiber and the CMOS imager are coupled together without using any magnification or minimization lenses. In a preferred version of this embodiment no lenses are used to couple the two and the fiber is simply adhered to the CMOS imager. In an alternate version of this embodiment a lensing system with near unity gain (e.g. in the range of 0.85× to 1.15× magnification) is used for the optical coupling. In addition to any other image processing stages the image processing hardware/software uses digital magnification techniques to increase the size of the resultant fiber image. Bilinear interpolation of the CMOS sensor's pixels, for example, would be a suitable image-scaling algorithm. This technique could be employed in tandem with embodiment 1 to further reduce the cost associated with fiber optic cameras. Note that this embodiment works particularly well with bundles that are constructed with relatively large fibers (e.g. greater than 10 micron), which are likely found in incoherent bundles. This is mainly due to the fact that CMOS imaging sensors typically have pixel sizes on the order of 1.5-5 micron squares. This means that no magnification is required to satisfy the Nyqvist requirement. Additionally larger CMOS pixels are typically less noisy than smaller CMOS pixels. This means that a larger fiber can be directly sampled (no magnification) with less noise than a smaller fiber. Fiber optic camera systems typically have relatively poor light acceptance so using a CMOS sensor with larger pixels is advantageous since they are typically less noisy and can be more sensitive with less noise.

There may be provided a method for directly visualizing a scene wherein an incoherent fiber optic bundle transports light from the scene to an imaging sensor. The data collected by the imaging sensor is shuffled according to a lookup table in order to recreate the original scene. At least the memory storing the lookup table and the fiber bundle are an independent subassembly such that the rest of the system can read the lookup table from the fiber assembly and process the image.

The lookup table may be calculated and stored during manufacturing and read during use.

Only a subset of the pixels corresponding to an individual fiber may be used during image manipulation.

The subset of pixels may be remapped according to the contents of the lookup table and an image of arbitrary size is realized by interpolating between the pixels.

Only a single pixel per subset may be used for remapping and said pixel is chosen by the system in order to mollify errors associated with the tolerance stack of the fiber-lens-sensor assembly.

One or more of a broken fiber map and relative fiber efficiency map may be also calculated and stored on the storage member.

The broken fiber map and efficiency map may facilitate image enhancement.

The method may be used to image objects such as a kidney stone or other urinary tract obstruction.

The method may be used for the identification of obstructions in other body lumens including, but not limited to the fallopian tubes, sinus, throat, and biliary system.

There may be provided a method of directly visualizing a scene wherein an incoherent fiber optic bundle transports light from the scene to an imaging sensor. The data collected by the imaging sensor may be shuffled according to a lookup table in order to recreate the original scene. The lookup table may be calculated as a calibration step immediately prior to use.

The method may be used for identifying a kidney stone or other obstruction in the urinary tract. A medical practitioner may perform the calibration step prior to use.

There may be provided a system for directly visualizing scenes, the system may include an incoherent fiber bundle and lens for transmitting light from the scene to an imaging sensor used to transform the light signal into an electrical signal, a lookup table used to reconstruct the original image by shuffling the image sensor pixels according to the data in the lookup table, and a processing member, which performs the shuffling.

The system may be used for directly visualizing a kidney stone.

There may be provided an apparatus for visualizing and optionally removing a kidney stone from a ureter wherein the device may facilitate in determining the relative location between it and the kidney stone using any method referred to in the specification.

The method may include using light spectroscopy.

There may be provided a method of identifying an object of interest in a body lumen wherein the method may include illuminating the scene with light, measuring the reflected and absorbed light in the scene by means of an imaging sensor and making inferences as to which objects are present in the scene based on the wavelengths absorbed and reflected.

The method may be used for identifying a kidney stone in the urinary track.

The method may include augmenting an image of a scene by detecting objects in said scene.

FIG. 8 illustrates a method 300 according to an embodiment of the invention.

Method 300 may include the following steps:

Stage 310 of directing light from an object, through at least one lens and a non-coherent fiber bundle and onto an imaging sensor. The non-coherent fiber bundle comprises multiple fibers. Each of the multiple fibers has a distal end and a proximal end. Stage 310 may include illuminating the object. The object may be located within a body lumen. The lumen may be part of the urinary tract such as the kidney, the bladder and the like. Stage 310 may include illuminating the object. Stage 310 may be preceded by a calibration stage of obtaining information about at least one of the transfer properties of the fibers, mapping information that associates between locations of distal ends and proximal ends of the multiple fibers and the like. The calibration stage may include imaging a calibration target and processing the received image to determine the information.

Stage 320 may include generating, by the imaging sensor, detection signals.

Stage 330 may include reconstructing at least a portion of an image of an object that faces the distal end of the multiple fibers; wherein the reconstructing is responsive to the detection signals and to mapping information that associates between locations of distal ends and proximal ends of the multiple fibers.

Stage 330 may include at least one out of the following stages:
a. Reconstructing the at least portion of the image in response to information about transfer properties of the multiple fibers.
b. Reconstructing the at least portion of the image in response to information about malfunctioning fibers of the multiple fibers.
c. Removing from a reconstructed image gaps between the multiple fibers. The removal of a gap formed between adjacent fibers of the multiple fibers may include interpolations between a subset of pixels out of all pixels associated with the adjacent fibers. The subset of pixels may include one or more pixels per fiber.
d. Digitally magnifying the image of the object.

Method 300 may also include stage 340 of responding to the image. For example removing an object from the lumen, guiding a medical procedure, updating information such as mapping information, information about at least one of the transfer properties and the like.

Method 300 may be executed in real time. Real time may indicate execution time of milliseconds or below. Real time execution of method 300 may allow a generation of a video stream of images without noticeable delay to the viewer.

The invention may also be implemented in a computer program for running on a computer system, at least including code portions for performing steps of a method according to the invention when run on a programmable apparatus, such as a computer system or enabling a programmable apparatus to perform functions of a device or system according to the invention. The computer program may cause the storage system to allocate disk drives to disk drive groups.

A computer program is a list of instructions such as a particular application program and/or an operating system. The computer program may for instance include one or more of: a subroutine, a function, a procedure, an object method, an object implementation, an executable application, an applet, a servlet, a source code, an object code, a shared library/dynamic load library and/or other sequence of instructions designed for execution on a computer system.

The computer program may be stored internally on a non-transitory computer readable medium. All or some of the computer program may be provided on computer readable media permanently, removably or remotely coupled to an information processing system. The computer readable media may include, for example and without limitation, any number of the following: magnetic storage media including disk and tape storage media; optical storage media such as compact disk media (e.g., CD-ROM, CD-R, etc.) and digital video disk storage media; nonvolatile memory storage media including semiconductor-based memory units such as FLASH memory, EEPROM, EPROM, ROM; ferromagnetic digital memories; MRAM; volatile storage media including registers, buffers or caches, main memory, RAM, etc.

A computer process typically includes an executing (running) program or portion of a program, current program values and state information, and the resources used by the operating system to manage the execution of the process. An operating system (OS) is the software that manages the sharing of the resources of a computer and provides programmers with an interface used to access those resources. An operating system processes system data and user input, and responds by allocating and managing tasks and internal system resources as a service to users and programs of the system.

The computer system may for instance include at least one processing unit, associated memory and a number of input/output (I/O) devices. When executing the computer program, the computer system processes information according to the computer program and produces resultant output information via I/O devices.

In the foregoing specification, the invention has been described with reference to specific examples of embodiments of the invention. It will, however, be evident that various modifications and changes may be made therein without departing from the broader spirit and scope of the invention as set forth in the appended claims.

Moreover, the terms “front,” “back,” “top,” “bottom,” “over,” “under” and the like in the description and in the claims, if any, are used for descriptive purposes and not necessarily for describing permanent relative positions. It is understood that the terms so used are interchangeable under appropriate circumstances such that the embodiments of the invention described herein are, for example, capable of operation in other orientations than those illustrated or otherwise described herein.

The connections as discussed herein may be any type of connection suitable to transfer signals from or to the respective nodes, units or devices, for example via intermediate devices. Accordingly, unless implied or stated otherwise, the connections may for example be direct connections or indirect connections. The connections may be illustrated or described in reference to being a single connection, a plurality of connections, unidirectional connections, or bidirectional connections. However, different embodiments may vary the implementation of the connections. For example, separate unidirectional connections may be used rather than bidirectional connections and vice versa. Also, plurality of connections may be replaced with a single connection that transfers multiple signals serially or in a time multiplexed manner. Likewise, single connections carrying multiple signals may be separated out into various different connections carrying subsets of these signals. Therefore, many options exist for transferring signals.

Although specific conductivity types or polarity of potentials have been described in the examples, it will be appreciated that conductivity types and polarities of potentials may be reversed.

Each signal described herein may be designed as positive or negative logic. In the case of a negative logic signal, the signal is active low where the logically true state corresponds to a logic level zero. In the case of a positive logic signal, the signal is active high where the logically true state corresponds to a logic level one. Note that any of the signals described herein may be designed as either negative or positive logic signals. Therefore, in alternate embodiments, those signals described as positive logic signals may be implemented as negative logic signals, and those signals described as negative logic signals may be implemented as positive logic signals.

Furthermore, the terms “assert” or “set” and “negate” (or “deassert” or “clear”) are used herein when referring to the rendering of a signal, status bit, or similar apparatus into its logically true or logically false state, respectively. If the logically true state is a logic level one, the logically false state is a logic level zero. And if the logically true state is a logic level zero, the logically false state is a logic level one.

Those skilled in the art will recognize that the boundaries between logic blocks are merely illustrative and that alternative embodiments may merge logic blocks or circuit elements or impose an alternate decomposition of functionality upon various logic blocks or circuit elements. Thus, it is to be understood that the architectures depicted herein are merely exemplary, and that in fact many other architectures may be implemented which achieve the same functionality.

Any arrangement of components to achieve the same functionality is effectively “associated” such that the desired functionality is achieved. Hence, any two components herein combined to achieve a particular functionality may be seen as “associated with” each other such that the desired functionality is achieved, irrespective of architectures or intermedial components. Likewise, any two components so associated can also be viewed as being “operably connected,” or “operably coupled,” to each other to achieve the desired functionality.

Furthermore, those skilled in the art will recognize that boundaries between the above described operations merely illustrative. The multiple operations may be combined into a single operation, a single operation may be distributed in additional operations and operations may be executed at least partially overlapping in time. Moreover, alternative embodiments may include multiple instances of a particular operation, and the order of operations may be altered in various other embodiments.

Also for example, in one embodiment, the illustrated examples may be implemented as circuitry located on a single integrated circuit or within a same device. Alternatively, the examples may be implemented as any number of separate integrated circuits or separate devices interconnected with each other in a suitable manner.

Also for example, the examples, or portions thereof, may implemented as soft or code representations of physical circuitry or of logical representations convertible into physical circuitry, such as in a hardware description language of any appropriate type.

Also, the invention is not limited to physical devices or units implemented in non-programmable hardware but can also be applied in programmable devices or units able to perform the desired device functions by operating in accordance with suitable program code, such as mainframes, minicomputers, servers, workstations, personal computers, notepads, personal digital assistants, electronic games, automotive and other embedded systems, cell phones and various other wireless devices, commonly denoted in this application as ‘computer systems’.

However, other modifications, variations and alternatives are also possible. The specifications and drawings are, accordingly, to be regarded in an illustrative rather than in a restrictive sense.

In the claims, any reference signs placed between parentheses shall not be construed as limiting the claim. The word ‘comprising’ does not exclude the presence of other elements or steps then those listed in a claim. Furthermore, the terms “a” or “an,” as used herein, are defined as one or more than one. Also, the use of introductory phrases such as “at least one” and “one or more” in the claims should not be construed to imply that the introduction of another claim element by the indefinite articles “a” or “an” limits any particular claim containing such introduced claim element to inventions containing only one such element, even when the same claim includes the introductory phrases “one or more” or “at least one” and indefinite articles such as “a” or “an.” The same holds true for the use of definite articles. Unless stated otherwise, terms such as “first” and “second” are used to arbitrarily distinguish between the elements such terms describe. Thus, these terms are not necessarily intended to indicate temporal or other prioritization of such elements The mere fact that certain measures are recited in mutually different claims does not indicate that a combination of these measures cannot be used to advantage.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. A medical imaging system comprising:
a non-coherent fiber bundle that comprises multiple fibers; wherein each of the multiple fibers has a distal end and a proximal end;
at least one lens optically coupled to the non-coherent fiber bundle;
an imaging sensor that is arranged to receive light received from the non-coherent fiber bundle and to generate detection signals; and
a non-volatile memory module that stores mapping information that associates between locations of distal ends and proximal ends of the multiple fibers.

2. The medical imaging system according to claim 1 wherein the at least one lens comprises a proximal lens that is optically coupled to the non-coherent fiber bundle.

3. The medical imaging system according to claim 1 wherein the at least one lens comprises a distal lens fixed to the non-coherent fiber bundle.

4. The medical imaging system according to claim 1 wherein the imaging sensor is adjacent to the non-coherent fiber bundle.

5. The medical imaging system according to claim 1 wherein the non-coherent fiber bundle and the at least one lens belong to a disposable portion of the medical imaging system.

6. The medical imaging system according to claim 1 wherein the non-volatile memory module also stores information about transfer properties of the multiple fibers.

7. The medical imaging system according to claim 1 wherein the non-volatile memory module also stores information about malfunctioning fibers of the multiple fibers.

8. The medical imaging system according to claim 1 further comprising an image processor that is arranged to receive the detection signals and the mapping information and to reconstruct at least a portion of an image of an object that faces the distal end of the multiple fibers.

9. The medical imaging system according to claim 8 wherein the non-volatile memory module also stores information about transfer properties of the multiple fibers; and wherein the image processor is arranged to modify the at least portion of the image in response to the information about transfer properties of the multiple fibers.

10. The medical imaging system according to claim 8 wherein the non-volatile memory module also stores information about malfunctioning fibers of the multiple fibers; and wherein the image processor is arranged to reconstruct the at least portion of the image in response to the information about malfunctioning fibers of the multiple fibers.

11. The medical imaging system according to claim 8 wherein the image processor is arranged to compensate for gaps between the multiple fibers.

12. The medical imaging system according to claim 8 wherein the image processor is arranged to compensate for a gap formed between adjacent fibers of the multiple fibers by performing interpolations between a subset of pixels out of all pixels associated with the adjacent fibers.

13. The medical imaging system according to claim 12 wherein the subset of pixels comprises a single pixel per fiber.

14. The medical imaging system according to claim 8 wherein the image processor is arranged to digitally magnify the image of the object.

15. A medical imaging system comprising: a non-coherent fiber bundle that comprises multiple fibers; wherein each of the multiple fibers has a distal end and a proximal end; at least one lens optically coupled to the non-coherent fiber bundle; an imaging sensor that is arranged to receive light received from the non-coherent fiber bundle and to generate detection signals; and an image processor that is arranged to receive the detection signals and reconstruct an image of an object that faces the distal end of the multiple fibers in response to the detection signals and to mapping information that associates between locations of distal ends and proximal ends of the multiple fibers.

16. The medical imaging system according to claim 15 wherein the at least one lens comprises a distal lens that is optically coupled to the non-coherent fiber bundle.

17. The medical imaging system according to claim 15 wherein the at least one lens is fixed to the non-coherent fiber bundle.

18. The medical imaging system according to claim 15 wherein the image processor is arranged to calculate the mapping information.

19. The medical imaging system according to claim 15 wherein the image processor is arranged to calculate the mapping information in response to information about malfunctioning fibers of the multiple fibers.

20. The medical imaging system according to claim 15 wherein the image processor is arranged to calculate the mapping information in response to an expected content of the image.

21-41. (canceled)
